(19) United States
(12) Patent Application Publication
(10) Pub. No.: US 2019/0216403 A1
(43) Pub. Date: Jul. 18, 2019

(54) METHOD FOR PREDICTING ACUTE AIRWAYS OBSTRUCTION OF A PATIENT WITH CHRONIC RESPIRATORY DISEASE

(71) Applicant: RESTECH S.r.l., Milano (IT)

(72) Inventor: Alessandro Gobbi, Bergamo (IT)

(21) Appl. No.: 15/869,246

(22) Filed: Jan. 12, 2018

(51) Int. Cl. A61B 5/00; A61B 5/085

(52) U.S. Cl. CPC A61B 5/7275; A61B 5/085

(57) ABSTRACT

A method for predicting the risk of acute airways obstruction of a patient j with chronic respiratory disease, comprising the steps of: taking an already available averaged (population-based) risk curve that quantifies the probability of having a future airways obstruction in at least future days; measuring the inspiratory resistance of one patient j for at least a prefixed number of days; calculating the probability of future acute airways obstruction of the patient j, adjusting the already available averaged (population-based) risk curve with a coefficient k that take into account the inspiratory resistance of the patient j.

### TECHNICAL FIELD

The present invention relates to a method for predicting acute airways obstruction of a patient with chronic respiratory disease. Chronic respiratory diseases, such as asthma and chronic obstructive pulmonary disease (COPD), are characterized by an underlying chronic inflammation of the lungs that leads to characteristic respiratory symptoms (dyspnea, cough, wheezing, etc.). Even during clinically stable periods, symptoms change frequently due to the complex dynamic pattern of the disease, to its progression over time and to the interaction of the respiratory system with external stimuli (viruses, air pollution, allergens, medications, etc.); such symptoms also worsen periodically towards acute events called exacerbations.

### BACKGROUND

Millions of children and adults worldwide are affected by chronic respiratory diseases: according to the World Health Organization (who.int/respiratory, accessed on November 2017) it is estimated that 235 million people currently suffer from asthma and 65 million have moderate to severe COPD. Their prevalence and related socio-economic costs are expected to rise in the upcoming years, further incrementing the already significant burden to the national healthcare systems.

Current management strategies of respiratory patients are aimed at stabilizing the disease, reducing its progression over time and the likelihood of future exacerbations. Some evidence indicates also that self-management programs made at home by the patient in combination with periodic ambulatory visits may improve patient management and contribute to better clinical outcomes (Powell et al., Cochrane Database Syst Rev, 2003).

### SUMMARY

At present, self-management programs are mostly based on the recording of symptoms on a daily basis. However, symptoms are susceptible to poor perception from the patients and to recall bias. Objective (quantitative) and personalized tools would be therefore desirable to be incorporated into novel management strategies to determine treatment changes and predict the course of the disease and future worsening on an individual basis before they manifest clinically by symptoms (Chung et al., Eur Respir J, 2017; Global strategy for asthma management and prevention, GINA, 2017). Among possible candidates, spirometry (or one of its output parameters, the peak expiratory flow, PEF) and forced oscillation technique (FOT) have been proposed for objective and personalized self-management programs of chronic respiratory diseases. Spirometry and PEF measurements have been extensively evaluated both in asthma and COPD but the results show that they are problematic in terms of accuracy if such recordings are done in unsupervised environments; moreover, due to the forced maneuver required, spirometry and PEF suffer from rapid decline in compliance from the patients.

Forced Oscillation Technique (FOT) is an alternative lung function test that has the potential to be used in self-management programs of both asthma and in COPD patients. FOT is a non-invasive method for investigating respiratory pathophysiology, requiring minimum collaboration by the patient during the measurement (Oostveen et al., Eur Respir J, 2003). In a typical FOT test, the patient breathes spontaneously through the device nozzle for a plurality of breaths while the device stimulates the respiratory system with a single or multiple frequency pressure stimulus (usually sine waves between 4 and 40 Hz) of low amplitude (usually 1-3 cmH₂O peak-to-peak) and simultaneously records the pressure and the airflow signals, usually at the patient's mouth. The ratio between pressure and flow represents the input impedance of the respiratory system (Zrs), which can be divided into its real (resistance, Rrs) and imaginary (reactance, Xrs) parts. Rrs and Xrs can be analyzed both in the time domain, along the breathing cycle (within-breath analysis of Zrs) and in the frequency domain (frequency analysis of Zrs). Rrs and Xrs can be further split into their inspiratory and expiratory components, i.e. into inspiratory and expiratory resistance (Rinsp and Rexp, respectively) and inspiratory and expiratory reactance (Xinsp and Xexp, respectively). FOT parameters are usually reported as their averages made using the measured plurality of breaths. Several methods are available from the literature to calculate within-breath impedance, including algorithms based on cross-correlation, fast Fourier transforms or least-squares (Kackza et al., Critical Reviews in Biomedical Engineering, 2011).

Even with the above limitations on the use of PEF in self-management programs, Frey and colleagues developed a method based on conditional probability to predict, at a population level, the risk of future exacerbations from the analysis of past variations in daily PEF measurements in a group of 80 asthmatic individuals (Frey et al., Nature, 2005). This method was then further extended by Thamrin and colleagues, who analyzed past variations in daily PEF measurements in two separate populations of 78 and 61 asthmatic individuals to calculate the risk of future exacerbations on an individual basis (Thamrin et al., JACI, 2011). Of note, this latter method required at least 64 daily PEF measurements for a proper initialization before calculating the first risk value; as new PEF values became available, the 64-day time window could be moved progressively over the patient's time-series and risk calculations repeated.

In a study by Gulotta and collaborators (Gulotta et al., Am J Resp Crit Care Med, 2012) a new conditional probability method to predict future acute airways instabilities based on past day-to-day airway variability of FOT parameters was tested on data from 10 mild asthmatic individuals who daily measured their Zrs at home. The main finding was that for these asthmatics the probability of occurrence of acute airways instabilities in a future time window W2 (prediction window) of at least 4 days could be predicted via an averaged risk profile derived from the same population, given the day-to-day variability of Rinsp measured in a past time window W1 of at least 4 days. Such probability profile is referred here as average conditional probability (ACP, FIG. 1).

This and other findings were object of the patent U.S. Pat. No. 9,668,673 that included the steps of: 1) identifying the presence of a chronic respiratory disease by FOT and 2) predicting future acute airways obstruction by FOT using the ACP method.

The step of identifying the presence of chronic respiratory disease can be carried out with several methods, manual or automated. The step of automatically predicting acute airway events in patients with respiratory diseases is summarized and incorporated herein. This step is achieved by means of a suitably programmed computer, which receives as input the measurements made on the patient, calculates the impedance values and the probability of future acute airways obstruction. The method uses a device able to measure mechanical respiratory parameters (Rrs and Xrs) by FOT. Technical details, the precision, stability and reliability of a suitable but non-exclusive device are described in Dellaca et al. (Dellaca et al., Physiol Meas, 2010). During the test the subject is required to wear a nose clip, and to support the cheeks with the hands while breathing spontaneously through the device nozzle for a plurality of breaths or for a predetermined amount of time (e.g., for two minutes in the study by Gulotta et al.). The method calculates the average within-breath FOT parameters of the measurement following the exclusion of breaths containing artifacts (cough, glottis closure, swallowing, etc.). For this purpose, the automatic algorithm described in Gobbi et al. (Gobbi et al., IEEE eTelemed, 2009) can be used. Alternatively, other manual, automatic or semi-automatic techniques can be used to this scope.

As an example for the ensuing description, FIG. 2 shows how to calculate the average Rinsp of a given measurement: within-breath Rrs at 5 Hz was estimated here from pressure and flow signals by the least-square algorithm (Kackza et al., Critical Reviews in Biomedical Engineering, 2011) using windows of 0.2 s (FIG. 2A). For each breath, which starting and ending points were identified from the zeroes of the flow signal, Rinsp was calculated by averaging the portion of Rrs contained in the central ⅗ of the inspiratory phase of the breath in question (bold segments in FIG. 2A). This procedure was reiterated on all breaths and the results from the breaths without artifacts were averaged to obtain the mean Rinsp of the measurement in question, thus resulting in one data point in FIG. 2B. The daily time series of Rinsp was built thereafter from these daily Rinsp values (FIG. 2B).

The prediction of future acute airways obstruction incorporated herein from patent U.S. Pat. No. 9,668,673 can be carried out by using the time-series of inspiratory or expiratory FOT parameters separately, the inspiratory ones being preferable. On any given day i, an acute airways obstruction was defined here as at least one value of Rinsp in the future W2 days, {i+1, i+2, . . . , i+W2}, above a predetermined threshold. Such threshold was set to twice the patient's predicted value of resistance, where the predicted value was determined from the reference equations reported in Pasker et al. (Pasker et al., Eur Respir Rev, 1994), using the patient's anthropometric data (age, gender, height and weight) reported in the Gulotta et al. study (Gulotta et al., Am J Respir Crit Care Med, 2012) as independent variables. On any given day i, the method quantifies the probability of acute airways obstruction in the future W2 days (W2>=4) given a today's FOT parameter measured for at least past W1 consecutive days (W1>=4). In the example reported in FIG. 1, this FOT parameter was the day-to-day variability of Rinsp, quantified by its standard deviation (SDRinsp, FIG. 2B) calculated in the past W1=8 consecutive days, {i−(W1−1), i−(W1−2), . . . , i}. Probability points were calculated as reported in the patent U.S. Pat. No. 9,668,673. Finally, the population-based probability curve (ACP, FIG. 1) was obtained by pooling together the probabilities calculated from 10 asthmatic subjects.

The present invention provides an automated method for predicting the risk of future airways obstruction based on FOT daily recordings that, compared with the population-based approach described in patent U.S. Pat. No. 9,668,673 (ACP FIG. 1) is applicable on an individual basis and that, compared with other personalized methods for prediction of acute airways obstruction (e.g. Thamrin et al., JACI, 2011), has the following improvements: 1) reduces the number of daily measurements required for the first prediction of risk (i.e. from 64 days of the Thamrin et al. method down to 4 days), and 2) increases the accuracy of risk estimation.

This and further objects are attained according to this invention by a method for predicting acute airways obstruction, in accordance with the accompanying claims.

### BRIEF DESCRIPTION OF DRAWINGS

Further characteristics and advantages of the present invention will be more apparent from the description of a preferred but non-exclusive embodiment according to the invention, illustrated by way of non-limiting example in the accompanying drawings, in which:

FIG. 1 is an example of an ACP curve that quantifies, at a population level, the probability of an acute airways obstruction given a today's pre-specified input FOT parameter;

FIG. 2 shows an example of a method for calculating the average Rinsp value from a FOT measurement and its day-to-day variability (SDRinsp);

FIG. 3 shows how the method described by Thamrin et al. (Thamrin et al., JACI, 2011) for calculating the probability of occurrence of future airways obstruction on an individual basis (named here individual conditional probability, ICP) was adapted to FOT measurements;

FIG. 4 shows the differences between the ICP and ACP probability values calculated from each available measurement in the time series of Rinsp of the 10 asthmatic individuals of the Gulotta et al. study (Gulotta et al., Am J Respir Crit Care Med, 2012) as a function of their SDRinsp.

In particular, in FIG. 1, it is showed an example of the ACP curve that quantifies, at the population-level, the probability of having an acute airways obstruction, in this example defined as at least one value of resistance greater than twice the subject's predicted value in a future W2 time window (in this example W2=7 days), given the today's day-to-day variability of a FOT parameter measured in a given W1 time window (in this example quantified by the standard deviation of inspiratory resistance, SDRinsp, in W1=8 past days).

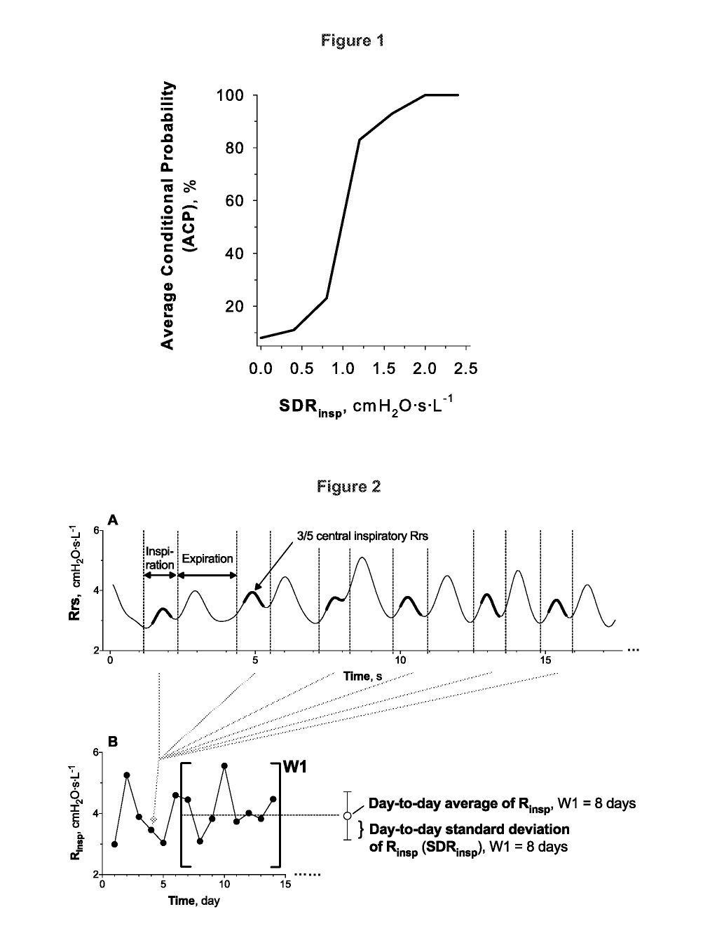

In FIG. 2, it is shown how, on any given day i, the SDRinsp is calculated from its correspondent daily time-series of Rinsp and within a given time window W1, {i−(W1−1), i−(W1−2},

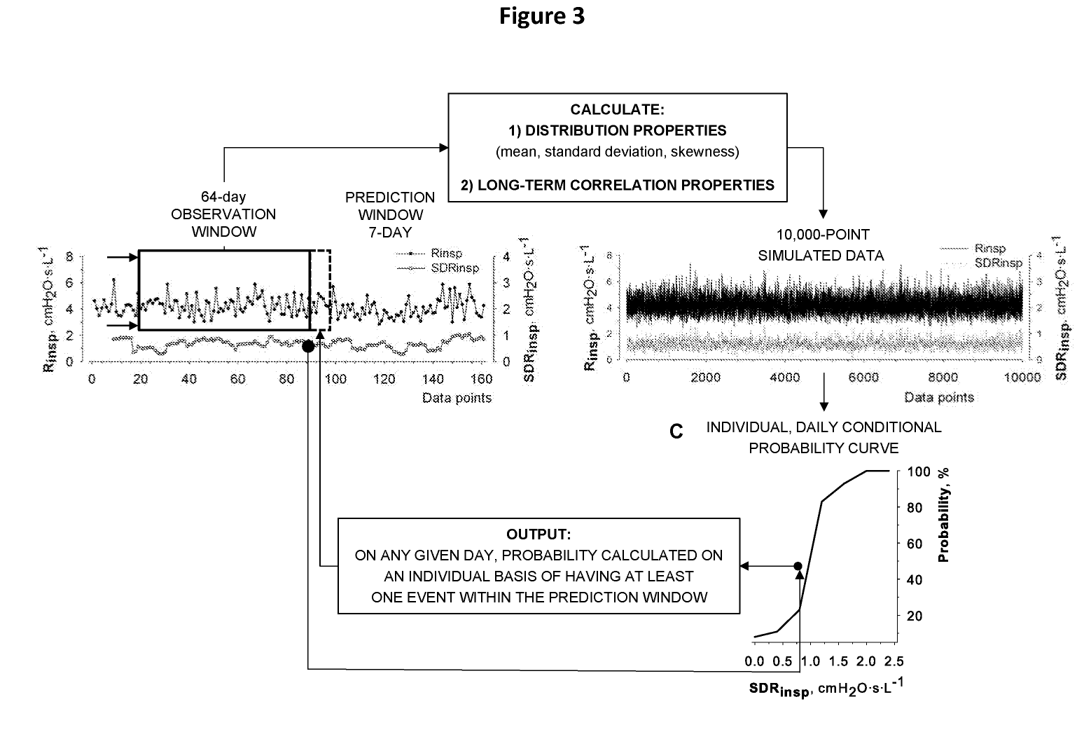

In FIG. 3, it is shown how the ICP method has been adapted to be used with FOT parameters. On any given day, from the original daily time-series of Rinsp, a new time-series of SDRinsp is generated by calculating the standard deviation of the Rinsp values over a W1=8-day time window moved progressively over the recorded data. Then, correlation properties and the first three moments of the distribution of Rinsp time-series are calculated over a 64-day time window as previously described for PEF time-series (Thamrin et al., JACI, 2011) and used to simulate two new time-series of 10,000 Rinsp and SDRinsp data points with the same properties as the original 64 data points. The two simulated time-series are then used to calculate a risk profile of having an acute airways obstruction (i.e. Rinsp above twice the subject's predicted value in the upcoming week, i.e. W2=7 days in this example) using the same method described in the patent U.S. Pat. No. 9,668,673, but in this case applied on an individual basis. Calculations are repeated over the simulated data, for every simulated data point, by moving this 64-day time window progressively over the simulated time-series. The outcome of this analysis is a risk profile that allows calculating, on any selected day and for the patient in question, the individualized probability of an acute airway obstruction occurring within the future W2 days (W2=7 in this example), given the patient today's day-to-day variability of the selected FOT parameter measured in W1 days, i.e. in this example given the today's SDRinsp calculated from the Rinsp values contained in a past window of 8 days containing also the current Rinsp value. Then, the 64-day window can be moved progressively daily over the patient's time series of Rinsp, and calculations can be repeated to obtain updated risk profiles.

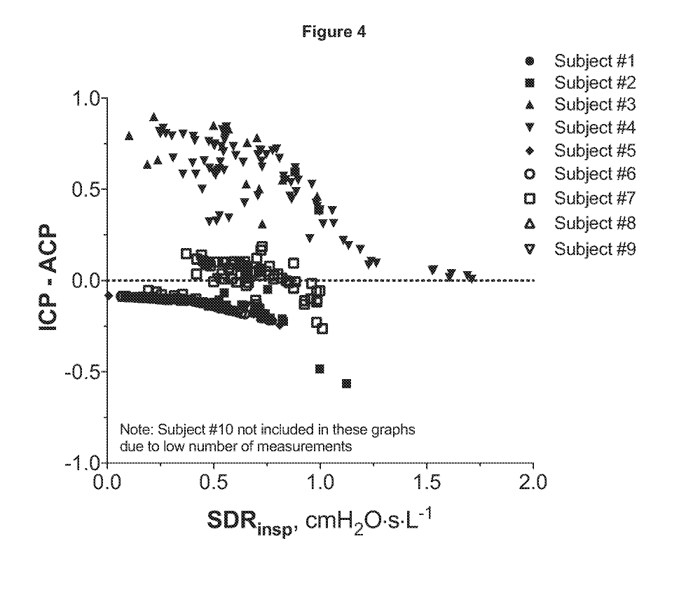

FIG. 4 shows the differences between the ACP (that quantifies the probability of future acute airways obstruction from a population-based risk profile applied to the patient in question) and ICP (that quantifies the same probability from an individual-based risk profile derived directly from the patient in question, see also the description of FIG. 3) values, calculated from the time series of Rinsp of 10 asthmatic individuals used to verify the results of the present invention (Gulotta et al., Am J Respir Crit Care Med, 2012) and plotted as a function of of SDRinsp. Such difference d_{j,i} for each individual subject j and for any given day i:

d_{j,i}=ICP(j,i)−ACP(j,i) (1)

was found to: 1) be proportional to difference between the patient's average Rinsp and his/her correspondent predicted value of resistance and 2) decrease with increasing SDRinsp values (FIG. 4).

### DETAILED DESCRIPTION OF THE INVENTION

From the above analyses, to improve the performance of the prediction, a correction factor k was derived to adjust the ACP curve object of patent U.S. Pat. No. 9,668,673, derived at a population level, with measures specifically related to the individual patient j who is currently making the FOT measurements. Accordingly, the population-based averaged risk curve ACP of FIG. 1 was modified to obtain the adjusted ACP_{ADJ} for the individual patient as follows:

$${ACP}_{ADJ , W 1 , W 2} ( j , i ) = {ACP}_{W 1 , W 2} ( i ) + {k}_{W 1} ( j , i ) , \\ {k}_{W 1} ( j , i ) = \frac{{R}_{insp , W 1} ( j , i ) - {Rrs}_{pred} ( j )}{{Rrs}_{pred} ( j )} \cdot [ 1 - {ACP}_{W 1 , W 2} ( i ) ] \qquad ( 2 )$$

### Where:

ACPAD1,W1,W2(j,i)=probability (risk) of future acute airways obstruction adjusted on an individual basis for the patient j on the day i;
ACPW1,W2(i)=on the given day i, probability of future acute airways obstruction in the future W2 days ({i+1, i+2, . . . , i+W2}) for the patient j, where such probability is calculated from said available averaged population-based risk profile given the day-to-day variability of Rinsp measured in the past W1 days ({i−(W1−1), i−(W1−2), . . . , i});
Rinsp,W1(j,i)=on the given day i, average day-to-day Rinsp of the patient j, calculated from said number of past days W1 (in this example W1=8 days, {i−(W1−1), i−(W1−2), . . . , i});
Rrs_{pred}(j)=predicted value of respiratory resistance, given the anthropometric characteristics of the patient j, i.e. the resistance of a healthy person with the same anthropometric characteristics as the patient in question; in this example, the Pasker et al. reference equations were used (Pasker et al., Eur Respir Rev, 1994) to determine the Rrs_{pred} from the following anthropometric parameters: age, gender, height and weight.

Instead of Rinsp and Rrs, other respiratory impedance parameters, measured at 5 Hz or at other frequencies, can be used in Equation (2), such as expiratory resistance (Rexp), inspiratory reactance (Xinsp), expiratory reactance (Xexp), total reactance (Xrs), etc. To quantify the improvement in accuracy of prediction of future airways obstruction of the present invention (ACP_{ADJ}) as compared with the previous population based risk profile (ACP) we studied the same group of 10 nonsmoking patients with mild asthma, and 10 nonsmoking, age-matched, healthy control subjects used to derive the ACP curve, object of patent U.S. Pat. No. 9,668,673. Subjects measured FOT data at 5 Hz daily at home in the morning for 6 consecutive months. We compared accuracy, sensitivity, specificity, and positive and negative predictive values between the ACP and ACP_{ADJ} methods in their ability to predict a future airways obstruction within the next 7 days (W2=7) for all the available data. Receiver operating characteristic curves were constructed by pooling together and separately the data from the 10 asthmatics and the 10 healthy subjects and by varying the cutoff points for the probabilities obtained from ACP and ACP_{ADJ}. The results showed that, in patients with asthma, acute airways obstruction were correctly predicted in W2 by the ACP_{ADJ} method with an accuracy of 94% (95^{th} confidence interval: 92% - 97%), a value significantly higher than ACP (75%, 95^{th} confidence interval: 68%-81%, p<0.001). At the cutoff point maximizing both sensitivity and specificity, identified using the J-statistics (Youden et al., Cancer, 1950), ACP_{ADJ} provided higher sensitivity (97% vs. 53%), comparable specificity (83% vs. 85%), higher positive predictive value (58% vs. 46%) and higher negative predictive value (99% vs. 87%) in predicting future airways obstruction events than ACP.

1. A method for predicting the risk of acute airways obstruction of a patient j with chronic respiratory disease, comprising the steps of:
a. taking an already available averaged (population-based) risk curve that quantifies the probability of having a future airways obstruction in at least future W2 days, given an input forced oscillation parameter measured for at least W1 past days during a plurality of respiratory cycles;
b. measuring the inspiratory resistance of the patient j for at least a prefixed number of days W1 at the same or different point in time than the measurements taken for calculating the said averaged risk curve;
c. calculating the probability of future acute airways obstruction of the patient j, for any day i, as following:
$${ACP}_{ADJ , W 1 , W 2} ( j , i ) = {ACP}_{W 1 , W 2} ( i ) + {k}_{W 1} ( j , i ) , \\ {k}_{W 1} ( j , i ) = \frac{{R}_{insp , W 1} ( j , i ) - {Rrs}_{pred} ( j )}{{Rrs}_{pred} ( j )} \cdot [ 1 - {ACP}_{W 1 , W 2} ( i ) ]$$
where:
ACP_{ADJ,W1,W2}(j,i)=probability (risk) of future acute airways obstruction adjusted on an individual basis for the patient j on the day i;
ACP_{W1,W2}(i)=on the given day i, probability of future acute airways obstruction in the future W2 days for the patient j, where such probability is calculated from said available averaged population-based risk profile given the day-to-day variability of Rinsp measured in the past W1;
R_{insp,W1(j,i)}=on the given day i, average day-to-day Rinsp of the patient j, calculated from said number of past days W1;
Rrs_{pred}(j)=predicted value of respiratory resistance, given the anthropometric characteristics of the patient j.

2. The method according to claim 1 characterized in that said averaged risk curve of having a future airways obstruction is calculated by identifying the presence of a chronic respiratory disease by FOT and predicting future acute airways obstruction by FOT using the ACP method.

3. The method according to claim 1 characterized in that said averaged risk curve has been calculated from a reference group of human subjects with chronic respiratory disease having similar severity/characteristics as the patient j in question during a plurality of respiratory cycles of said human subjects;

4. The method according to claim 1 characterized in that said prefixed number of days W1 is >=4 days.

5. The method according to claim 1 characterized in that said prefixed number of days W2 is >=4 days.

6. The method according to claim 1 characterized in that the average inspiratory resistance (Rᵢₙₛₚ) from a plurality of respiratory cycles is measured at the frequency of 5 Hz.

7. The method according to claim 1 characterized in that the day-to-day average inspiratory resistance (Rᵢₙₛₚ) is measured for W1>=4 days.

8. The method according to claim 1 characterized in that the input forced oscillation parameter used to calculate the averaged (population-based) risk curve of having a future airways obstruction and used to calculate the ACP_{ADJ} for the said patient j is the reactance.
